(19) United States
(12) Patent Application Publication
(10) Pub. No.: US 2022/0111206 A1
(43) Pub. Date: Apr. 14, 2022

(54) Cochlear Implant Retention and Concealment Bow

(71) Applicant: Catherine Elizabeth Garner, CARY, NC (US)

(72) Inventor: Catherine Elizabeth Garner, CARY, NC (US)

(21) Appl. No.: 17/497,031

(22) Filed: Oct. 8, 2021

Related U.S. Application Data
(60) Provisional application No. 63/090,400, filed on Oct. 12, 2020.

(51) Int. Cl. A61N 1/36; A61N 1/372; A45D 8/14

(52) U.S. Cl. CPC A61N 1/36038; A45D 8/14; A61N 1/37235

(57) ABSTRACT

The invention pertains to the restriction of movement of one or two cochlear implant processors affixed inside of a decorative hairbow. The functionality of a concealed pocket within the hairbow is to limit the movement of a cochlear implant processor while concealing the presence of the processor. Concealed pockets are located bilaterally on the hairbow to provide users with either a unilateral or bilateral wearing option. The hairbow is affixed to a retainment item that can be attached to the user's hair or head. Due to the decorative nature of the hairbow, young cochlear implant users may be more inclined to wear the cochlear device.

### CROSS-REFERENCE TO PRIORITY APPLICATION

This application hereby claims the benefit of pending U.S. Provisional Patent Application No. 63/090,400 for a “Cochlear Implant Retention and Concealment Bow” (filed Oct. 12, 2020 at the United States Patent and Trademark Office), which is hereby incorporated by reference in its entirety.

### FIELD OF THE INVENTION

The present invention generally relates to the retainment and concealment of a cochlear implant processor.

### BACKGROUND

The prevalence of congenital sensorineural hearing loss, >40 dB HL (Hearing Loss), is about 0.002% of live births. The prevalence of hearing loss increasing with age is supported by a 1998 Center for Disease Control study concluding that approximately 3% of school-age children (grades 3,6, and 9) are found to have a form of unilateral or bilateral hearing loss. There nave been many studies that provide evidence that school-age children with hearing loss experience a multitude of auditory, education, and psychosocial problems. Increasingly so, as compared to his or her hearing peers, a child with hearing loss may exhibit difficulty understanding speech in noisy conditions, localizing sounds, developing age-appropriate language and cognitive skills, and performing tasks in educational settings. Even with early diagnosis, many children with hearing loss may still experience low self-esteem, anxiety, difficulty maintaining peer relationships, and decreased social support networks.

Self awareness progresses in stages as a child goes from birth through childhood. Young children navigate their environment by noticing similarities and differences and how it relates to their previous experiences. A child with a cochlear implant may notice his or her outward appearance differs because of the device on his or her ear. Because the need to “fit in” increases as children get older, individuals with cochlear implants may begin to purposefully not wear the device even though it improves hearing. Concealment of the cochlear implant may help a child to fit in amongst their peers, improve their self esteem, and encourage the child to wear the cochlear implant more often.

### SUMMARY

During strenuous activity, it is a common occurrence that a cochlear implant processor worn behind the ear will fall off of an individual's head. As the processor is attached via a cable to the magnet, the entire cochlear implant will dislodge and fall off. This presents the following problems:
(1) The user is now rendered partially or completely deaf.
(2) The cochlear implant can be damaged in falling to the ground.
(3) The user is unable to complete the present activity.

According to the present invention, the cochlear implant retention and concealment bow is able to both retain and conceal a cochlear implant processor in a fashionable and less obtrusive form factor.

The bow serves to restrict the movement of the processor, so it does not come off of the user's head while doing strenuous activities. This is achieved through a pocket that is concealed within one or more loops of the bow.

The user will place the bottom of the cochlear implant processor into the bottom pocket of the bow ensuring the cable and attached magnet remain out. The user will then slide the ear hook through the top loop (if applicable). Next, the user will ensure that the microphone openings are not impeded by any part of the bow and place the ear hook on the outside of the bow.

The user is able to choose the bow's material and specific design. Additionally, a user has the options to add embellishments. This is all in effort to allow the user to feel more comfortable and in control of wearing the cochlear implant processors.

The bow serves as a way to not only reflect one's own personality and fashion, but also retain and conceal the cochlear implant processor from direct view. Particularly with young children and adolescents, the idea of concealing the processor might entice the user to reliably and regularly wear the cochlear implant devices. A cochlear device is not helpful when it is not worn by the user. Evidence suggests that wearing just one processor when an individual is bilaterally impaired significantly reduces hearing fatigue.

The bow serves an additional function of restricting the movement of the processor, so it does not come off of the user's head while doing strenuous activities.

### BRIEF DESCRIPTION OF THE DRAWINGS

FIG. 1 is an exemplary depiction of bilateral cochlear implant processors placed within the cochlear implant retainment pocket and concealing loop(s).

FIG. 2 is an exemplary depiction of the location of the pocket(s) used to restrain the cochlear implant processor.

FIG. 3 is an exemplary depiction of the loop(s) concealing the presence of the cochlear implant processor.

FIG. 4 is an exemplary depiction of two example affixable items that can be used to attach the present invention onto a user's head or hair.

### DETAILED DESCRIPTION

The present invention describes a device to retain and conceal a user's one or two cochlear implant processors.

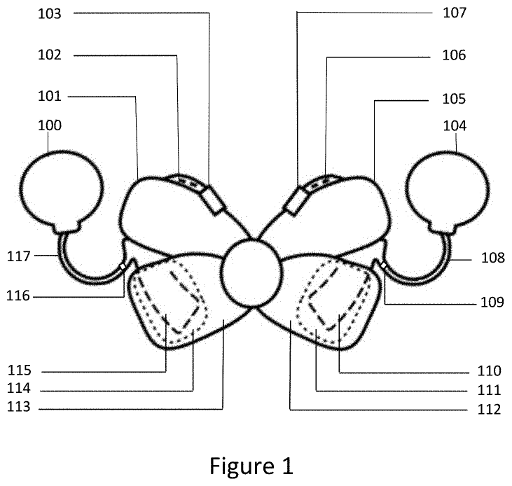

FIG. 1 is an exemplary depiction of bilateral cochlear implant processors placed within the cochlear implant retainment pocket and concealing loop(s). For the bilateral user, the Left Cochlear Implant Processor (115) and the Right Cochlear Implant Processor (110) are placed inside of the corresponding Left Pocket (114) and Right Pocket (111). The Left Coil Cable Connector (116) and the Left Coil Cable (117) as well as the Right Coil Cable Connector (109) and the Right Coil Cable (108) are situated between the Left Bottom Bow Loop (113) and the Right Bottom Bow Loop (112) respectively.

As depicted in FIG. 1, the top portion of the Left Cochlear Implant Processor (115) and the top portion of the Right Cochlear Implant Processor (110) are placed into the Left Top Bow Loop (101) and into the Right Top Bow Loop (105) respectively. The Left Microphone Openings (102) and the Left Microphone (103) are situated at the top of the Left Top Bow Loop (101) as to be unimpeded by the material. The Right Microphone Openings (106) and the Right Microphone (107) are situated at the top of the Right Top Bow Loop (105) as to be unimpeded by the material. The Left Magnet (100) is then affixed to the user's internal receiver. The Right Magnet (104) is then affixed to the user's internal receiver.

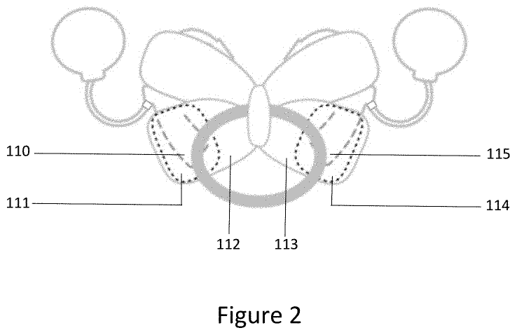

FIG. 2 is an exemplary depiction of the location of the pocket(s) used to restrain the cochlear implant processor. The drawing presented is of the underside view of the present invention. For the bilateral user, the Left Cochlear Implant Processor (115) and the Right Cochlear Implant Processor (110) are placed inside of the corresponding Left Pocket (114) and Right Pocket (111).

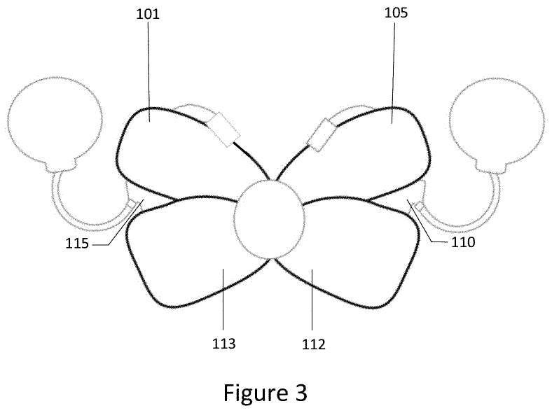

FIG. 3 is an exemplary depiction of the loop(s) concealing the presence of the cochlear implant processor. The Left Bottom Bow Loop (113) and the Left Top Bow Loop (101) conceal the Left Cochlear Implant Processor (115). The Right Bottom Bow Loop (112) and the Right Top Bow Loop (105) conceal the Right Cochlear Implant Processor (110).

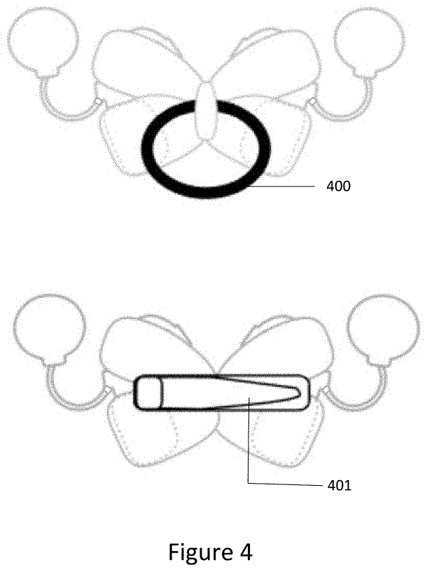

FIG. 4 is an exemplary depiction of two example affixable items that can be used to attach the present invention onto a user's head. Different hair fasteners are used to suit the specific needs of the user. Two common examples of hair fasteners are an elastic hair band, Hair Fastener Option (400), or a barrette, Hair Fastener Option (401).

1. A hairbow apparatus to be worn by a user comprising: at least one loop fashioned in the shape of a bow, wherein said loop contains a first pocket for retention and concealment of at least one cochlear implant processor.

2. The invention of claim 1 further comprising a second loop fashioned in the shape of a bow, wherein said second loop contains a second pocket for retention and concealment of a second cochlear implant processor.

3. The invention of claim 2 further comprising temporary or permanent closure of the edge of said first or second loops to create said first or second pockets.

4. The invention of claim 3 further comprising a plurality of loops fashioned in the shape of a bow, wherein said plurality of loops provide additional retention and concealment of at least one cochlear implant processor.

5. The invention of claim 4 further comprising openings in said first, second, or plurality of bow loops to provide unimpeded acoustic path to said first or second cochlear implant processor microphones.

6. The invention of claim 4 further comprising temporary or permanent openings in said first, second, or plurality of bow loops to provide routing of said first or second cochlear implant coil cables.
